(19) United States
(12) Patent Application Publication
(10) Pub. No.: US 2019/0391253 A1
(43) Pub. Date: Dec. 26, 2019

(54) Thermal Biomarker System

(71) Applicant: Harold Szu, Bethesda, MD (US)

(72) Inventor: Harold Szu, Bethesda, MD (US)

(21) Appl. No.: 15/191,367

(22) Filed: Jun. 23, 2016

Related U.S. Application Data
(60) Provisional application No. 62/183,511, filed on Jun. 23, 2015.

(51) Int. Cl. G01S 13/86; G01J 5/00; A61B 5/01; A61B 5/05; A61B 5/00

(52) U.S. Cl. CPC G01S 13/867; G01J 5/0025; A61B 5/0077; A61B 5/0507; A61B 5/0075; A61B 5/015

(57) ABSTRACT

A passive thermal spectral imaging camera includes an active confocal tunable microwave illuminator tuned to human physiology bio-molecules, and can also include an active microwave radar and a passive electro-optic-infrared camera. The camera can also include a thermal biomarker, which can include a microwave oven, a microwave-mixer, and a beam expander. The beam expander can include a metallic pin-hole array. An imaging system includes a passive electro-optic-infrared camera, and a thermal biomarker, and can also include a microwave oven, and a microwave mixer. The thermal biomarker can include a confocal tunable microwave illuminator, which can include a waveguide coupled to a magnetron of the microwave oven and to the microwave mixer. The microwave mixer can be configured to downshift and/or upshift an output of the magnetron.

### CROSS-REFERENCE TO RELATED APPLICATION

This is related to, and claims priority from, U.S. Provision Application for Patent No. 62/183,511, which was filed on Jun. 23, 2015, the entire disclosure of which is incorporated herein.

### FIELD OF THE INVENTION

The invention relates to imaging systems.

### BACKGROUND OF THE INVENTION

There are over 8 million aging baby boomers of the age of 70 or older in the U.S. alone. In their next decade of life this population will likely suffer from one of four major disorders: type II diabetes, stroke, heart attacks, or some form of cancer. These disorders will continue to add stress to the U.S. health care system, resulting in inevitable quality of service reductions. The potential increase in workload will push hospitals and care givers to their limits in terms of financial hardship and individual workload. These conditions will increase the potential for human error and fatigue, further exacerbating the situation. Immediate proactive measures are required to avoid further reduction in quality of care. A decade ago the former NIH director Elias Zerhouni advocated the points of care with a $2B budget for a new approach, Personalized, Preemptive, Preventative, Participatory (4-P) principles, for faster turnaround “from the bench to the bed.” One of the goals of such points of care has been to move the patient from a hospital sick bed patient to a homecare environment with hospital day visits.

### SUMMARY OF THE INVENTION

The invention improves the less-discriminating passive thermal spectral imaging camera through the use of a noninvasive and noncontact but active Confocal Tunable Microwave Illuminator (CTMI) targeting at human physiology bio-molecules in order to increase the performance in terms of the receiver operation characteristics (ROC), plotting the probability of detections (PD) versus the false alarm rate (FAR).

A non-linear synergism between active microwave radar and passive EO-IR cameras for non-real time Stokes upshift from radar giga (10⁹) Hz to Optics peta (10¹⁵) Hz. This teaching improves the specificity of infrared cameras, and the early shortfalls of a somewhat uncomfortable & non-tunable chill-biomarker (c-BM) with CTMI targeting at type II diabetics insulin, stress generated hormones, and adrenaline for heartbeats and blood pressure, as well as cortisone/adrenaline for glucose energy utilization.

Thermal biomarker (T-BM): The invention includes a modified affordable and ubiquitous microwave oven operated at 2.4 GHz; λ≈12 cm. with a commercial microwave-mixer for upshift or downshift, and a beam expander CTMI that is made of a metallic pin-hole array. Such a system, connected from the back of the microwave oven with a table top tunable microwave mixer, and a beam expander, aperture Dₚᵢₙₕₒₗₑₛ≈20 cm to improve the 2^{nd} wavelet source uniformity in the Rayleigh resolution≈λ_{mw}/Dₚᵢₙₕₒₗₑₛ) for safety reasons.

Following FDA/CDRH type-3 devices, the microwave oven power-squared-range product (PRP) vs. the time-on-target (TOT) are the material properties for applications from healthcare to defense. Radar and EO-IR can be mixed non-linearly at airport TSA surveillance points, or at a critical perimeter for a second look to differentiate animals from human beings.

The invention features a realistic physical display based on Boltzmann entropy theory and irreversible thermodynamics. A weakly-linear thermal imaging backscattering data model \vec{X}(f_{IR}/t,\vec{x}) convolutes the unknown impulse response matrix propagation-lens [A(f_{IR,MW}|t,\vec{x})] with unknown target entropy sources \vec{S}_{bb} (f_{MW}/t,\vec{x}). The Blind Sources Separation (BSS) is solved using the Lagrange Constraint Neural Network (LCNN) learning weight matrix[W]\vec{X}≈\vec{S} satisfying the Helmholtz Minimum Free Energy (MFE) derived from irreversible thermodynamics ΔS>0: such that ΔS=ΔS_{RV}+ΔS_{bb}>0. According to the conservation of energy law ΔQ_{RV}=TₒΔS_{RV}=−ΔE_{bb}. It is determined that the optimization cost function min. H_{bb} ≡E_{bb}−TₒS_{bb}≤0. A consistent BSS solution sought ΔH=\vec{μ}. {[W]\vec{X}−\vec{S}_{bbo}}≤0. The physics display follows the Boltzmann entropy definition S=k_{B} Log W yielding the Maxwell-Boltzmann canonical probability

$$W = exp ( \frac{{s}_{RV}}{{k}_{B}} ) = exp ( \frac{{S}_{RV} {T}_{o}}{{k}_{B} {T}_{o}} ) = ( - \frac{{E}_{bb}}{{k}_{B} {T}_{o}} )$$

where the zero-boundary eigen-states Eₙ=Σₙ₌₀^{∞}(n+½) [symbol] ω defined the Planck radiation spectral curves of black box (BB) parameterized by Kelvin temperature T. Given the Wien's displacement law λₘₐₓT≈2.8 mm ° Kelvin derived at the zero slope, each entropy source can be associated with an equivalent Kelvin temperature

$$T = \{ {T}_{o} + \frac{Δ}{2} , {T}_{o} , {T}_{o} - \frac{Δ}{2} \} ,$$

e.g. Tₒ≈37°. In summary, the DIR spectral ratio per pixel change detection for human malignant versus benign tumors discrimination is solved, by noninvasive CTMI TM, displayed in visible red (cold body), green (medium benign) to blue (hotter malignant).

The invention improves a traditional dual infrared spectrum camera using electrical but non-cryogenic cooled sensor material. The camera is designed with a low dynamic range apps (8 bits) for near field of view (FOV) and limited pixel on target (POT), in terms of MWIR (PbSe) and LWIR (VOₓ); etc.

According to an aspect of the invention, a passive thermal spectral imaging camera includes an active confocal tunable microwave illuminator tuned to human physiology bio-molecules.

The camera can also include an active microwave radar and a passive electro-optic-infrared camera.

The camera can also include a thermal biomarker, which can include a microwave oven configured to operate at about 2.4 GHz., a microwave-mixer configured for upshift and/or downshift, and a beam expander. The beam expander can include a metallic pin-hole array, which can include apertures having a diameter of about 20 cm.

According to another aspect of the invention, an imaging system includes a passive electro-optic-infrared camera, and a thermal biomarker.

The system can also include a microwave oven, and a microwave mixer. The thermal biomarker can include a confocal tunable microwave illuminator, which can include a waveguide coupled to a magnetron of the microwave oven and to the microwave mixer.

The microwave mixer can be configured to downshift and/or upshift an output of the magnetron.

### BRIEF DESCRIPTION OF THE DRAWINGS

FIGS. 1(a) and 1(b) show aspects of confocal tunable microwave illumination: FIG. 1(a) shows a tunable microwave oven operated at the typical magnetron frequency to target the resonant frequency of thermal-biomarker (T-BM); FIG. 1(b) is a graph of an ROC curve resulting from the device tunable at the lower end of intrinsic resonance molecular frequencies bands for penetration reasons.

FIG. 2 shows surrogate human samples made of protein (raw egg without shell) and water.

FIGS. 3(a)-(l) show balloon images with different wrapping material to model and simulate cancers inside the body.

FIG. 4(a) is an illustration of a microwave heating water molecules.

FIG. 4(b) is a diagram of an adrenaline molecule.

FIG. 4(c) is a diagram of a cortisol molecule.

FIG. 4(d) is a diagram showing the imaginary part of the dielectric constant plotted against the temperature and the penetration depth at two microwave frequencies.

FIG. 5 is a diagram of the Planck black body radiation spectral density.

FIG. 6 is a diagram showing analytic continuation selective projection of desired targets by means of millimeter wave radar CTMI in terms of Boltzmann entropy theory along the Planck spectral curve for any portion of measuring spectral shape to determine the unique Kelvin temperature per spectral curve.

### DETAILED DESCRIPTION OF THE INVENTION

The present invention supports a 4-P program with affordable homecare device physics. A noninvasive and noncontact inexpensive biomarker (BM) is applied to support a passive and ordinary infrared (IR) spectral band camera. This design is a new thermal BM (T-BM) delivered by a uniform microwave beam from a confocal tunable microwave illuminating (CTMI) device linked from a microwave oven, pointed at targets to track malignant versus benign tumors, as well as to discover stress hormones at a checkpoint line-up (responsible for the phenomenology of increased heart rate, blood pressure, sweating, and energy production). The word “confocal” is taken from Minsky, who designed in 1965 at the object source plan with pin-holes array for increasing the sharpness resolution beyond the Rayleigh criterion. The 2 bands/2 cameras should be combined into a single camera, electrically but non-cryogenically cooled, in the near-field small pixels on target (POT) for easy co-registration with smartphones and PCs to every home-alone-senior (HAS). The system can have dual usage for both early warning homecare for aging baby-boomers and others, and as a homeland security surveillance device. The false alarm rate (FAR) can be reduced by the tunable non-contact CTMI as T-BM as well as a user-friendly physics display (as opposed to computer vision VR or AR) that can help discriminate disorders and pets from human, as well as alerting to other hazards such as when a caregiver that does not show up as expected.

In summary, the headstone proclaiming S=k Log W marks the gravestone of Ludwig Boltzmann. It turned out that similar to the Rosetta stone having decoded the Hieroglyphs in terms of the Greeks, surveillance sensing in the full electromagnetic spectrum may be decoded by the gravestone in terms of visible RGB color pixels as optical flow to human visual system (HVS). This is possible because the unknown targets are modeled as if the scattering (backward, for active probing; forward, for passive probing) in terms of Planck temperature-unique radiation spectral curve, which allows us analytically to continue nonlinearly along the temperature-unique Planck family of curves, for example, from microwave (cooking water≤3 GHz, 10 cm) to the visible thermal color display (RGB 0.8, 0.5, 03 μm) (too active or too hot would be more bluish than reddish). The Planck spectral curves are applied as the consistent approximation that did not imply the unknown targets physically to be black boxes, nor gray boxes; a consistent yard stick is introduced as the metric for the optical display. The radiologist might someday apply CTMI to excite molecular electrons of hydrogen bonds to the screen and track the physiology novelty change detection in terms of molecular electron vibrations at increasing dielectric constant of denser tumors, instead of the traditional x-ray imaging (XRI) that disturbs the deep nucleon proton charges with long-term accumulative memory effect at the genetic DNA level.

The Boltzmann headstone is chosen as the Rosetta stone, because the left-hand side entropy S as the sensing measuring the degree of uniformity (for example, the starlight scatters off atmospheric molecular turbulence), then the right-hand side

$$W = exp ( \frac{S}{{k}_{B}} ) = exp ( - \frac{E}{{k}_{T} T} )$$

turns out to imply the canonical ensemble to derive the Planck spectral curves. Moreover, Neal's 3^{rd} Law of thermodynamics predicted a positive and non-zero Kelvin temperature T>0; T≠0 that inferred an irreversible thermodynamics ΔS>0 for collision mixing toward totally uniformity in a closed system, that could no longer support life as the heat death asserted by Boltzmann.

Engineering apps must have consistent approximations. To derive the Planck temperature-unique radiation spectral curve as the display metric, the conservation of thermal energy change ΔQ_{bb}=TΔS_{bb}=−ΔE_{bb}:

$${W}_{bb} = exp ( \frac{{S}_{bb} T}{kT} ) = exp ( - \frac{{E}_{bb}}{kT} )$$

is applied to derive the so-called Maxwell-Boltzmann canonical probability. Given the black box (defined by zero boundary condition), Planck solved discrete standing wave eigenstates E_{bb}={Eₙ}; Eₙ≡(½+n) [symbol] ω; ω≡2πf. Together, they followed the canonical partition function Q_{bb}=\sqrt{z}Σₙ₌₀^{∞}; z≡exp(−=ℏω);

$$β ≡ \frac{1}{kT} .$$

Then, an average with the ensemble of all possible internal energy

$$< {E}_{bb} {>}_{{Q}_{bb}} = dos \cdot [ \frac{h ω}{exp ( \frac{h ω}{kT} ) - 1} ] ≡ {U}_{Planck}$$

yielded the celebrated Planck radiation spectral with density of states

$$dos ≡ \frac{8 {πω}^{2}}{{\vert {C}_{o} \vert}^{3}} .$$

In summary, given the multi-spectral sensing data \vec{X}_{bb}, LCNN is applied, solving BSS for a set of equivalent BB targets. The own specific value, slopes, and shapes a set of Kelvin temperatures T's for each BB target were estimated from each. As a result, the analytical continuation could be applied for each entropy source along the temperature-unique Planck spectral curves always toward the RGB color temperature display for any sensing probing frequency.
Background Information: Leveraging Homeland Security with Home-Front Healthcare for Seniors

An innovation was given, based on the nonlinear synergistic coupling in the human body with microwave illumination upon human stress hormones, and two passive imaging using IR filtering on a smartphone: one for first-look cueing and the other for second-look confirmation after the CTMI. This protocol for public healthcare watch will be taken for early warning of those home-alone seniors who have (1) a smartphone and (2) a microwave oven (at 2.45 GHz at 12 cm wavelength) (the “haves”) and those who do not have them (the “have nots”). The household microwave oven must be furthermore modified with a wave-guide to a table-top tunable mixer at the bio-molecular spectra, and a confocal pin-hole array for microwave beam expansion, of: (1) insulin of diabetics II, (2) adrenaline of heart attacks, (3) cortisol/adrenaline of strokes, and (4) massive tumor growth of cancers, which are among the top four killers of the 8 million aging baby boomers beyond 70 years old in their next decade or more living in the US alone. Moreover, the disaster remediation program must screen the ordinary population, including those who do not have a smartphone and microwave oven, to prevent epidemics.

Passive sensing is usually performed with EO-IR (electro-optics for day/infrared for night) cameras sampling forward scattering of stellar radiation sources. Active sensing, usually done in defense surveillance, exploits radio frequency (RF) full electromagnetic EO-IR-RF spectra backscattering from objects against their swarming robotic nature, say UXV (X=Air, Marine, Ground), camouflaged by natural weather in foggy, raining, cloudy, snowing conditions, or man-made fire and smoke clutter or other jamming sources. Passive optic sensing is affordable, but less discriminative at the molecular levels at a distance. Active radar sensing is expensive, yet less dense in pixel structure. Synergistically combining EOIR sensing nonlinearly with RF sensing provides useful surveillance AiTR with better ROC curves (for example, increase PD and reduce FAR). Apps are homeland defense at perimeters, and the healthcare for home-alone seniors for early warning of type II diabetes, stokes, heart attacks, and cancers.

A working hypothesis about the growth of cancers is that all cancers share nonstop growth in anaerobic energy production (phenomenological speaking, the Warburg effect is to ferment neighboring tissue, rather than going originally through those healthy energy outsourcing mitochondria, cells within our cells). As a result, the tumors tend to have a dense medium through rapid and persistent growth that requires active proteomic profiling for cancer biomarker discovery. Toward this phenomenology, introduce a class of nonintrusive and noncontact biomarkers (BM) referred to as thermal biomarkers (T-BM) are introduced to prompt “haves” to see the physician. For simplicity, the noninvasive T-BM is divided into (a) chill biomarker (c-BM), called “neo-angiogenesis BM”, that is, the tumor-supply capillary beds that were built in a hurry without the usual muscular linen controlled by autonomic nerve system (ANS) that will be contracted when immersing two hands in icy-cold water (the so-called leaky faucet c-BM); (b) Density increases the heat capacity using thermal biomarker illuminating with tunable microwave to warm up the bio-tissue filled mostly with water, etc., known as CTMI as a noncontact T-BM.

Experiments with Surrogate Materials

The passive EOIR (day and night) cameras are augmented with a T-BM to enhance the receiver operation characteristics (ROC). The T-BM is the CTMI that has a waveguide led from the microwave oven magnetron (2.45 GHz, and λ˜12 cm) and followed with microwave mixers that can tune to T-BM characteristic frequencies that were either downshifted toward L-band (1 GHz), or up-shifted to S-band (2˜4 GHz) to X-band (10 GHz). These are useful for the stress hormone and dense tumor mass T-BM. The full electromagnetic spectrum must be physically used for real-world sensing and display in RGB color optics for Human Vision System (HVS) for Aided Target Recognition (AiTR); for example, DHS developed Terra Hz as very long IR to see through jackets & walls.

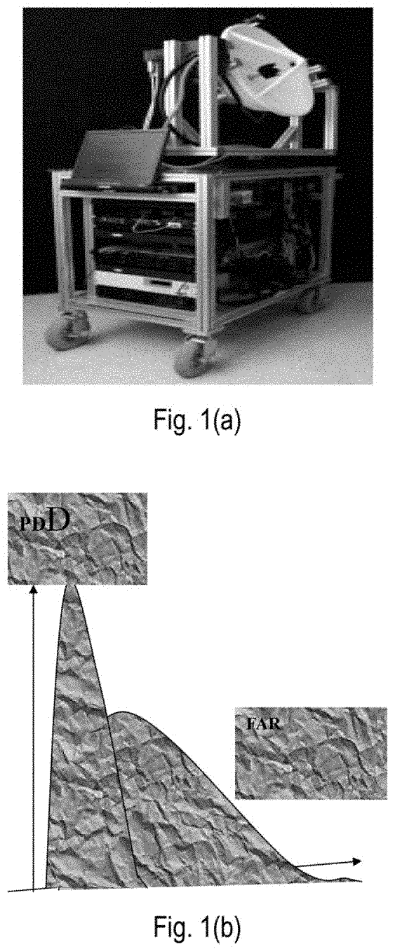

As shown in FIG. 1, if CTMI is applied in 1(a) that is tunable from the microwave oven operated at the typical magnetron (2.45 GHz@λ=12 cm) to target the resonant frequency of the T-B) that is tunable at the intrinsic resonance molecular frequencies bands of which the lower end is chosen for penetration reasons AiTR has a better ROC (left) curve shown in 1(b).

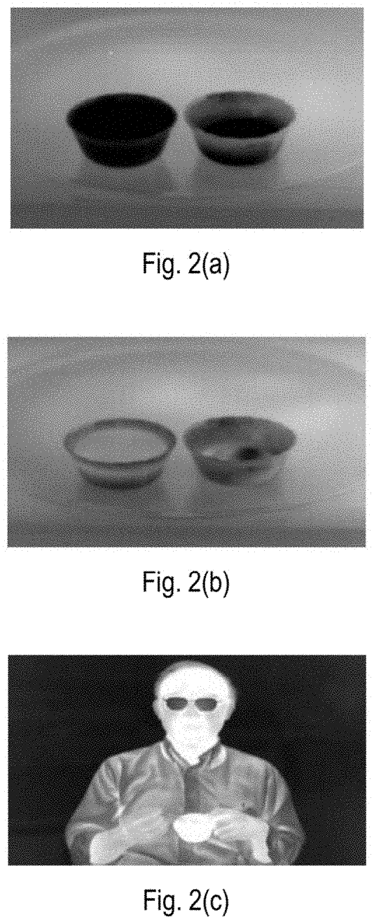

As shown in FIGS. 2(a)-(c), surrogate human samples are chosen that are made of protein (raw egg without shell) and water (as humans are 60% made of water). The microwave oven radiation source is at 1500w @ 2.45 GHz; Infrared camera is made of Mikron-Camera: model 7640 at LWIR, 8-14 μm, 640×480 VOX Detector; Distance: 1.5 m.

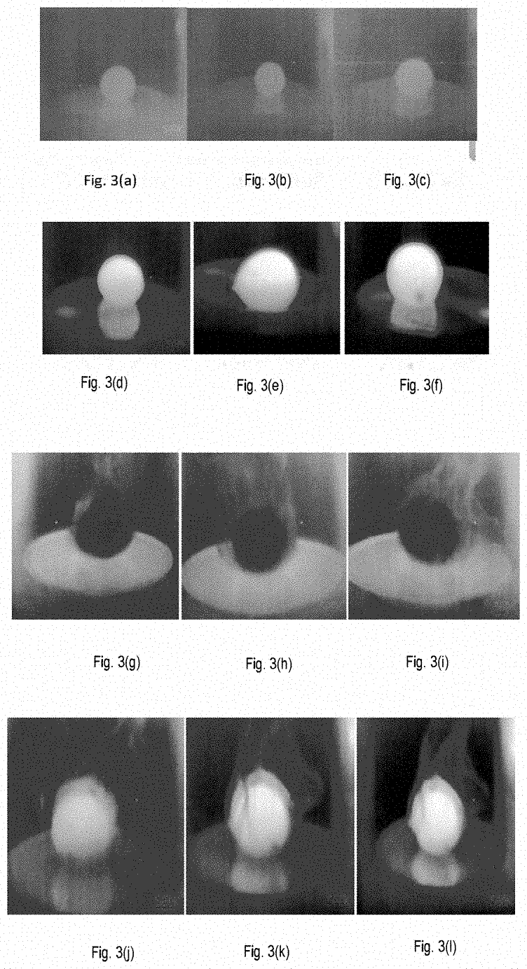

FIG. 3 illustrates the results of penetration depth testing. Different wrapping material was chosen to model and simulate cancers inside the body. Two images of two balloons, one without bag coverage 111 g heated up faster, the other of 115 g heated up less in the same 1 second over 15 seconds. Lesson Learned: Data indicates that microwave penetration is an important factor.

Balloon 1: No bag covers, initial mass of 111 g;

Balloon 2: Inside 2 thin garbage bags, initial mass of 115 g

| Time (s) | B1 Temp (° C.) | B2 Temp (° C.) |
|---|---|---|
| 0 | 27.5 | 26.3 |
| 1 | 27.8 | 26.5 |
| 2 | 28.5 | 26.8 |
| 3 | 31.1 | 27.8 |
| 4 | 32.9 | 28.8 |
| 5 | 35 | 29.4 |
| 6 | 36.5 | 33.6 |
| 7 | 36.9 | 31.3 |
| 8 | 42.9 | 39 |
| 9 | 45.2 | 42 |
| 10 | 49.6 | 44.3 |
| 11 | 55.7 | 49.9 |
| 12 | 60.1 | 47.3 |
| 13 | 66.4 | 41.6 |
| 14 | 71.3 | 59.9 |
| 15 | 78.8 | 69.9 |

The invention leverages defense resources, especially those which have obvious dual usages. For example, the synergistic combination of the EO_IR imaging and the RF bio-marker may be viewed as a time-delayed Strokes down-shifted (or anti-Strokes up-shifted). The display in both usages will be based on the Boltzmann entropy law S=k_{B} Log W and the irreversible thermodynamics law ΔS>0. These sensing and displaying technologies could be useful for homeland security and home front defense, especially at critical perimeter checkpoints, and aging home-alone seniors (“haves”) facing type II diabetes, strokes, heart attacks, and some forms of cancers. Several important dual usages and challenges are itemized as follows:
(1) Beyond near field Communication: smartphones and PCs have communication requirements similar to UXV, for example, over the horizon (OTH) and line of sight (LOS) to map along the geodesic horizon, providing an effective but less vulnerable wireless relay system in case they fall in the hands of an adversary;
(2) Management & Acquisition: cost-effective analysis of acquisitions comparing large quantity of UAV Tier II (man-size) to one giant Global Hawk having a limited geodesic coverage;
(3) Efficient Strategy: massive parallel UXV having a rich variety of strategies, including swarm usage that can combine, enhance, and synergize capabilities on the battlefield, whether for communications, target acquisition, lethality, etc.;
(4) Surveillance: at the critical perimeters that are mobile missile launchers, nuclear submarine cruising mobility;
(5) Health Care: home front surveillance for have-nots.

Irrespective of the duel usage in nonlinear synergistic combination of full electromagnetic spectrum for surveillance for either homeland or home fronts, the common requirement to optically display in realistic color will be the focus, according to Boltzmann entropy physics, rather than by the popular computer vision making belief virtual reality (VR) of augmented reality (AR).

Using a simple rule of thumb, because the speed of light is a constant in a vacuum Cₒ=λf=2.99×10¹⁰=29 cm×10 Hz, then 1 GHz is associated with about 1 foot wavelength, or one third of a meter. And 100 GHz-frequency systems used for automobile collision avoidance in bad weather is about 1% of a 1-foot wavelength, that is, 0.3 mm, which is the lower end of the sub-mm-wave microwave regime. Beside just heating water molecules, hydrogen bonds at 18 GHz, a microwave oven prefers 2.45 GHz for the tradeoff of lower frequency penetration into volume food, which is a short enough wavelength to propagate in the straight line of sight (LOS), and yet long enough to go around all sizes of particulates (1 μm diameter droplets are known empirically to be best for staying airborne longest). To increase the specificity of the affordable passive dual-band infrared (DIR) imaging system for AiTR, a noncontact microwave RF-T-BM is adopted for the increase of probability of detection (PD) together with the reduction of false alarm rate (FAR), to differentiate clothed humans from hairy animals, the T-BM at stress hormones could be targeted, especially for home alone seniors (HAS), who often have midnight crises such as heart attacks or strokes. Using T-BM and DIR, the midnight crisis might be mitigated or prevented altogether. Due to pre-waking increase of physiologic activity, a first look of DIR might detect warming up with increasing infrared spectral MWIR/LWIR ratio that should be immediately interrogated using a tunable RF T-BM targeted at facial bare skin blood capillary for measuring secretion of wakening adrenaline hormones. If further increase in DIR spectral ratio occurs, then the seniors shall be prompted to immediately massage themselves and loosen up muscular constrictions to help blood circulation prior to waking and getting out of bed.

Four generic challenges differentiating targets from clutters in Home Alone Seniors:
(i) Diabetic type II, that is, insulin secretion spatiotemporal timing issue,
(ii) Tumorigeneses have higher order of magnitude for menopausal females at ductile carcinoma in-situ (DCIS) breast cancers, in cases of nulliparous effect (a la Singapore Epidemiology); also prostate cancers at a slower growth rate for senior males. For example, cellular differentiation at different parts of our body varies, nothing in in-situ biopsy and cellular growth rate performed in the Petri dish can replace a physician's diagnosis-based truth. However, even the biopsy and Petri dish approaches have their limitations as well. These limitations include uncertainly regarding where and how many biopsy samples per 3-D tumor are required and also physiologic and morphological constraints in the human body for cell differentiation and cell apoptosis that in vivo frequently tracks in vitro growth (in dishes) is different than in vivo growth (in life) because of these physiological boundary conditions. A cheaper faster better safer (CFBS) T-BM screening for breast tumors by DIR can be taken frequently to track Tumorigeneses, that is, tumor development, and effective treatments of the specific quantitative prescription of chemotherapy. The invasive x-ray mammogram gold standard is only useful to confirm at the belated last 4^{th} stage with the metastasis micro-calcification. The nonspecific harmless DIR with the help specific noninvasive biomarkers T-BM & c-BM can augment the x-ray last stage with the early-stage screening based on the neo-angiogenesis & intra-vasation.
(iii) Midnight crisis of heart attacks and strokes that might be due to wake-up stress hormones (adrenalin for heartbeat and blood pressure & cortisone for energy production) that should be specifically targeted by CTMI for stress hormone as bimolecular T-BM.
(iv) Likewise, homeland security Transportation Security Administration (TSA) might apply proactively noncontact silent interrogation of line-up passengers at airport checkpoint without the slow down and the embarrassment to determine psychologically the stress hormones to separate sweating experienced frequent travelers passengers carrying illicit substances hidden inside their cavities;
(v) Trigger-wire digital fence surveillance system required to reduce far at the critical perimeter separating humans from animals or newly buried UXO.

The full electromagnetic (EM) spectrum is exploited, EO_IR_RF, for distant sensing, avoiding atmospheric propagation obscurants; furthermore, nonlinear synergistic EM interaction is exploited upon the target itself, which can generate a unique target feature set for AiTR. For example, when the targets are human, unique human stress hormones are exploited that were generated from the brain center called the hypothalamus as the commander in chief (CIC) that governs body temperature, thirst, hunger, sleep, circadian rhythm, moods, sex drive, and sets off the pituitary alarm system, as CEO, that further prompted through the autonomic nervous system (ANS), Vagus nerve through spinal cord, the adrenal glands atop kidneys to release adrenaline as the messengers, carried in the bloodstream and affecting the Autonomous Nervous System (ANS), which controls heart rate, dilation of the pupils, and secretion of sweat and saliva, and cortisone (a 21-carbon steroid hormone released by the adrenal gland in response to stress to suppress the immune system, thus reducing inflammation and attendant pain and swelling at the site of the injury for glucose energy production). If the CTMI is tuned specifically (away from 2.4 GHz cooking water molecules) at these specific stress hormones, an alarm light can be shined on the object together with CTMI T-BM to detect specific emotional responses releasing these stress hormones, and then a second DIR look can image the motion-detected object to ascertain if the response was from a human (that is, not animal or otherwise).

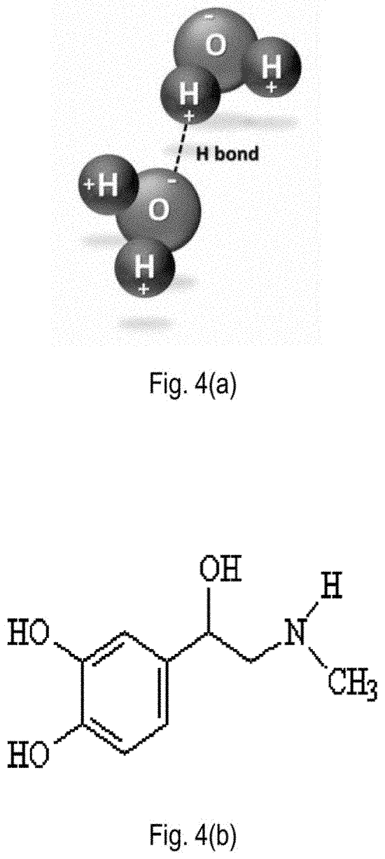

Microwave energy heats water molecules (see FIG. 4(a)), and adult humans are composed of more than 60% water. FIGS. 4(b) and (c) show adrenaline and cortisol molecule, respectively. FIG. 4(d) shows the imaginary part of the dielectric constant plotted against the temperature (° C.) and the penetration depth in cm, at two microwave frequency 2.45 & 5.8 GHz. For a pure water solution with no ions, it should be noted that the relaxation time constant of the imaginary dielectric constant for heating is optimum at 18 GHz, not the 2.45 GHz of typical microwave ovens. This is due to penetration depth of volume cooking. Grant et al. have established a linear relationship between the relaxation time of water and the molecular weight of 20 biological molecules with molecular weights up to 68000 (hemoglobin), which suggests that the size of the molecule is the predominant influence.

The common technology exploits the Artificial Neural Network (ANN) unsupervised learning in Blind entropy-Source Separation (BSS) without knowing the sensor lens antenna and medium transfer functions. The precise optical display is necessary for household application of microwave imaging. Thus, the Boltzmann molecular entropy is introduced: S=k_{B} Log W as the Rosetta headstone to relate the sensing to the display. The irreversible thermodynamics relation ΔS>0 is also introduced, understood from molecular physics that ever-increasing degree of uniformity owing to incessant molecular collision mixing in an isolated system. The sensing is provided by the integration of the entropy S as the measure of degree of uniformity of those target-scattering cross sections and the display in by the photon collision canonical phase space volume

$$W ≡ exp ( \frac{{ST}_{o}}{{k}_{B} {T}_{o}} ) = exp ( - \frac{nh}{λ {C}_{o} {k}_{B} {T}_{o}} ) ,$$

where Maxwell replaced by the conservation of energy the loss of reservoir thermal energy STₒ with the gain of internal subsystem photon energy

$$\{ {E}_{n} = ( n + \frac{1}{2} ) ℏ ω = ( n + \frac{1}{2} ) h / λ {C}_{o} \vert n = 1 , 2 , . . . \} .$$

The realistic display of RF is possible, according to the analytic continuation along the Planck radiation spectral curve at unique temperature of source. Planck is derived from Maxwell-Boltzmann canonical probability. The inverse sources problem is guesstimated by minimum Helmholtz free energy that is derived from the Boltzmann irreversible thermodynamics (ΔS>0). Once one can determine the Kelvin temperature associated with the radiation sources, one can analytically continue along the Planck radiation spectral curve from microwave lengths to optical wavelengths, and project to RGB display.

### Review of Medical Imaging

As WWII baby boomers with increasing life span enjoy longevity, they also have a higher prevalence of developing chronic illnesses such as strokes, heart attacks & carcinomas as Home Alone Seniors (HAS). Increased funding for imaging research can have beneficial applications in both homeland security at checkpoints and homecare scanning provided that imaging systems are CFB and safer for an aging population. Sensing front ends can include (1) electromagnetic (EM) transversal waves (EO IR RF), (2) acoustic longitudinal waves (that is, sonar, ultrasound), or (3) solenoid loop magnetic field lines (that is, undersea navigation, f-MRI).

As of recently, medical imaging is no longer done using a single modality, but rather as a combination of modalities. As such, co-registration of an optical display becomes even more important for on-going R&D activities. For example, there is a 200-times higher rate of metabolic glycolysis of energy production (into pyruvate kinase enzyme, giving rise to the Nobel Laureate Otto Warburg effect) associated with cancer cells that also results in morphologic changes. Traditionally, diagnosis is made using positron (e⁺) emission tomography (PET), which annihilates tissue electrons, producing a pair of γ-rays via radioactive marker modified hexokinase substrate. PET scans are increasingly read alongside CT or magnetic resonance imaging (MRI) scans, revealing metabolic information about blood hemoglobin usage with oxygen (diamagnetic) and without oxygen (ferromagnetic). This is done with the combination, co-registration so-called “PET-CT” or “PET-MRI” (FIG. 2) so that areas of abnormality on the PET imaging can be more perfectly correlated with anatomy on CT or MRI images. This is useful for moving organs or structures with higher anatomical variation outside the brain. For example, at Forschungszentrum Jülich GmbH Institute ($0.5B, thousands of scientists, 500 Ph. D. students), as a member of Helmholtz Association of German Research Centers associated with Aachen University, PET imaging is combined with 9.4-Tesla magnetic resonance tomography (MRT). Likewise, the invention also combines confocal microwave imaging with dual infrared spectral (DIR) imaging in a close coupling sense, resulting in microwave illumination that will produce a “warm biomarker’ to enhance imaging of dense and faster-growing malignant tumors having a large heat capacity.

Hospital x-ray laboratory screenings for bone density in senior adults are usually electrically generated by a high-voltage x-ray tube. This is slightly different from nuclear medicine using nuclear isotopes, say iodine, generating nuclear radiation gamma rays etc. having a wavelength ranging from 0.01 to 10 nanometers. Detection is based on the micro-calcification deposit (left behind by cell death and seen as black dots in the x-ray screen and film). In contrast to x-ray and nuclear medicine approaches, the invention frequently and conveniently applies harmless microwave imaging to track tumor development and tumor treatment for home-alone seniors in an aging society. This low energy (˜1 eV and less in the microwaves) can harmlessly perturb molecular outer shell electrons as the Maxwell displacement dielectric current vibrations without raising tissue temperature.

The CFBS practice of public healthcare follows the 4P Modern Medicine Practices, proposed by former NIH director Elias A. Zerhouni, which will be more Predictive, Personalized, Preemptive, and Participatory (4P). For cancer detection, there are at least 4 stages of tumorigenesis, each requiring different specific 4P Practices and utilizing specific stimulus and response less-invasive tracking of biomarkers (or tracers in nuclear medicine) for individual treatment of malignant carcinoma. These 4 stages of cancers include:
(1) Primary Tumor Invasion Stage: lacking a specific imaging modality, a good candidate is molecular-tagged functional florescence imaging, or functional confocal microwave imaging.
(2) Intravasations Stage: early detectable malignant tumor at mm scale has been demonstrated by means of dual infrared multispectral imaging (for example, a modified NVESD 3^{rd} Gen camera in a narrow close-up field of view) by comparing two multi-spectral pictures. One picture is taken before and the other is taken after a cold challenge (for breast Tumorigeneses by immersion of two hands in icy cold water). The comparison can help radiologists differentiate a newly-built blood-supply capillary without muscular linen that does not contract under a cold challenge. This abnormal mechanism is known as the neo-angiogenesis, so-called “leaky faucet” effect (by Hoekstra & Szu, et al. 2009 & 2014).
(3) Extravasations transport stage: lacking a reliable imaging modality, a good candidate might be f-MRI. For example, blood hemoglobin oxygen utilization in a reversible majority-voting rule between red blood cell hemoglobin having ferromagnetic property (without attachment to oxygen at multiple iron sites) and hemoglobin with diamagnetic property (with majority iron sites attached with oxygen molecules). This is known as the hemodynamic such that f-MRI can track oxygen utilization for certain growth functionality.
(4) Metastasis stage: tumor cells are dead and leave behind micro-calcification used for ion current communication detectable by x-ray imaging, for example, mammogram.

One candidate imaging device to illustrate the importance of a man-in-loop display for malignant tumor screening is Confocal Tunable Microwave Imaging (CTMI). This modality is based upon detection of abnormally larger dielectric constants for tumors than for normal tissue.

The trade-off of the image sharpness at the focal plane by microwave illumination at the conjugate object plane through a set of fine pin-hole arrays that are in contact with the subject's skin. This early design of CTMI becomes safer because of the recent advent of nano-pulse magnetrons. Microwave becomes a safer oven oscillating molecular surface electrons near 2.4 GHz (˜12 cm wavelength) reversibly. Unfortunately, pin-hole arrays at the object plane require long exposure times and may result in motion blur at the object cellular level.

Applying microwave energy results in thermal vibration of molecular electrons of water molecules at 2.4 GHz. With the advent of nano-pulses, an efficient dipole bowtie antenna, and a confocal microwave illuminating infrared imaging (CMIII) configuration (by limiting illuminating point sources through pin-holes geometry with long exposure, few minutes), to produce uniformly flat heating sources, according to the Huygens wave-front wavelets propagation principle. The microwave wavelength is short enough to travel along the line of sight (LOS) and is a safer reality provided one can build in motion blur compensation. Someday, one might augment and eventually replace the x-ray-based mammograms used for breast cancer detection. While mammograms are highly efficient with low-dosage x-ray exposure, accumulated damage due to excitation at atomic nucleons level result from repeated exposure. For repeated assessment and screening application, a non-ionizing imaging system for assessing tumorigenesis is desirable. The invention contributes (1) a theory of CTMI radar illuminating with optical DIR visual display; (2) automatic motion blur compensation to correct for wave-front tilts by using a self-reference matched filtering concept (cf. Szu, “local instances of good seeing”), namely the piecewise long exposure centers are used to re-register the short exposures with their local centers (having wrong wave-front tilts, but correct spatial frequency content) (Szu, JOSA, Opt Com. 1994). Furthermore, it may satisfy the CFBS principle for a potential tissue carcinoma screening. It is anticipated that a homecare CTMI with automatic de-blur will become as popular as a kitchen microwave oven for tracking a malignant tumor for treatment if a CFBS practice version of CTMI is realized. At each antenna position, the bowtie or dipole antenna is excited with an ultra-wideband pulse. The received backscatter signals are processed by a data-adaptive algorithm that removes the artifact caused by antenna reverberation and back scatter from the skin-breast interface, followed by 3-space-time beam forming to image backscattered energy as a function of location. With malignant-to-normal dielectric contrasts down to 1.5:1 for a 4-mm synthetic tumor at G Hz broad band[17,18]. Recently, Paul Meaney et al. discovered the complexity of breast cancer due to the breast surface skin thickening [20].

### Boltzmann Entropy & Irreversible Thermodynamics Theory of Display

Whereas active sensing would be backscattering, passive sensing is a forward scattering. Irrespective of active or passive, it is desirable to build an intelligent consistent display for a man-in-the-loop Aided Target Recognition (AiTR) system to sense through a natural obscurant or artificial jammer. For this, a distant sensing, active or passive, is assumed to be weak. Thus, the measurement system can be modeled as the linear impulse response theory of unknown mixing matrix [A]:

\vec{X}(f|t,\vec{x})=[A]\vec{S}(f|t,\vec{x}) (1)

A mixture of multiple-source components is denoted as an order vector set \vec{S}={Sᵢ|i=1, 2, 3, . . . }

Serendipitously, the Boltzmann molecular scalar entropy component Sᵢ turns out to be the Rosette stone for a consistent display job:

Sᵢ=k_{B} Log Wᵢ, (2)

which provides a consistent measure of each i-th component involving the sensing integration of scattering cross sections, over each uniform components. For example, a mountain-top rock may have more paleontology information than eroded beach sands, which possess identical molecules but much less internal energy details but larger degree of uniformity measured as the Boltzmann molecular entropy (as opposed to the Shannon measure of a communication channel capacity).

### Inverse Sources Problem by Means of Minimum Helmholtz Free Energy

Irreversible thermodynamics,

ΔS>0, (3)

asserts the ever-increasing degree of uniformity owing to incessant molecular collision mixing in an isolated system (according to Neal's 3^{rd} law of thermodynamics that as indeed NIST did not reach the absolute zero Kelvin temperature). Historically speaking, Ludwig Boltzmann had a serious argument about Eq (3) with Henri Poincare, who insisted on the time-reversal invariant of Newtonian dynamics:

$$m \frac{{d}^{2} \vec{x}}{{d ( \pm t )}^{2}} = \vec{F} .$$

At the end of his life, Boltzmann committed suicide and willed to mark with his gravestone with his conviction (S=k Log W), without his family name. It turns out that the resolution of the Boltzmann-Poincare dilemma is that the Newtonian dynamics proving the irreversible thermodynamics is more than the equation, but also the initial boundary conditions, that are never time reversible invariant.

The isothermal sensing environment on the earth may be generally modeled as the reservoir (RV) while the target sub-systems for display purpose in an equivalent yard stick of set of black boxes (BB) at different Kelvin temperatures (that does not mean the target has literally the black color, but rather zero Von Neumann boundary condition used to derive the eigen-states). Since

ΔS=ΔS_{RV}+ΔS_{bb}>0; then −ΔS=−ΔS_{RV}−ΔS_{bb}≤0.

Although the exact energy of the reservoir is not known, nor that of the subsystem targets, their losses and gains must be balanced. Thus, the Helmholtz minimum free energy principle (without the none-usable random energy the energy that is left to do the work, in a Carnot engine sense) can be derived by conceptually integrating over all the scattering cross-sections

H_{bb}≡E_{bb}−TₒS_{bb}≤0 (4)

where the change of unknown reservoir heat energy ΔQ_{RV}≡TₒΔS_{RV} is replaced with the change of unknown target internal energy −ΔE_{bb}. Because the system impulse response function is unknown, the inverse problem is blindly solved using an artificial neural network (ANN) learning matrix, denoted by a bold-faced letter in bracket: [W]≈[A]⁻¹ to guesstimate the answers in Blind Sources Separation (BSS) seeking at the MFE:

$$Δ {H}_{bb} = ( \frac{Δ {H}_{bb}}{\vec{Δ S}} ) \cdot \vec{Δ S} = \vec{u} \cdot \{ [ W ] \vec{X} - {\vec{S}}_{bbo} \} \to 0. \qquad ( 5 )$$

known as Lagrange (energy slope \vec{μ}) Constraint Neural Net (LCNN) (Szu & Hsu for remote sensing [2], & early screening of carcinoma, Szu, Miao & Qi [3]; Szu et al. U.S. Pat. No. 7,366,564 (Apr. 29, 2008); U.S. Pat. No. 7,355,182 (Apr. 8, 2008)). That LCNN BSS proved for the first time at MFE the empirical bi-linear rule postulated by Neurobiologist Donald Hebb more than a half century ago:

$$\frac{δ \{ {W}_{i , j} \}}{δ t} = \frac{δ H}{δ \{ {W}_{i , j} ]} = {\vec{μ}}_{i} {\vec{X}}_{j} , \qquad ( 6 )$$

where the Lagrange energy slope

$${\vec{μ}}_{i} = ( \frac{Δ {H}_{bb}}{\vec{Δ S}} )$$

turns out the so-called house-keeping glia cells to be the “missing half-Einstein brain,” one per neuron, as there are 10 billion neurons and 10 billion glia cells. The thermodynamic learning rule is to keep the brain temperature at the isothermal equilibrium in the direction of MFE slope [4].

CTMI & DIR optical imaging will be reviewed from the classical supervised LMS Wiener noise filtering and Vander Lugt matched filter as well as modern unsupervised Lagrange Constraint Neural Network (LCNN), which overcomes the curse of the dimensionality of limited library templates in the L2 norm sense and compressive sensing of linear excitation coefficients in the sensing or not L1 norm sense. Toward that, it is derived (a) from the LHS of Boltzmann entropy equation to the Helmholtz free energy minimization for solving the inverse BSS entropy problem and (b) from the RHS of the Boltzmann entropy equation to the Planck radiation spectral for direct Image entropy-Source color-Temperature (IST) problem for the display.

The rigorous sensing and display protocol requires 3 steps as follows: (1) solving point-by-point the inverse BSS entropy problem from multi-spectral sensing at individual pixels; (2) fitting the Planck spectral curve to determine the associated Kelvin temperature from the shape of spectral curvature; and (3) applying an analytical continuation along the specific Planck spectral curve to RGB micro-meter (μm) mapping for optical flat screen display.

### Direct Source Display Problem by Analytical Continuation of Planck Radiation Spectrum

To derive the Planck spectral curve, the 1^{st} law of thermodynamics is combined with the Boltzmann entropy law in terms of an equivalent probability phase volume known as Maxwell-Boltzmann (MB) canonical probability:

$$W = exp ( \frac{{S}_{RV}}{{k}_{B}} ) = exp ( \frac{{S}_{RV} {T}_{o}}{{k}_{B} {T}_{o}} ) = ( - \frac{{E}_{bb}}{{k}_{B} {T}_{o}} ) ,$$

A relationship between energy and absolute temperature (Kelvin) is derived from the 1^{st} and the 2^{nd} laws of thermodynamics (that is, the loss of reservoir thermal energy −ΔQ≡−TₒΔS=ΔE, is the gain of subsystem internal energy and integrated with the zero boundary conditions).

To reproduce the Planck radiation spectral data, it is assumed that an isothermal set of black boxes in the environment reservoir with EM standing waves represented by eigenstates E=Σₙ₌₀^{∞}Eₙ; Eₙ=(½+n) [symbol] ω and ω≡2πf. Consequently,

$$W \to W ( {E}_{n} ) = exp ( - \frac{{E}_{n}}{kT} ) = \sqrt{z } {z}^{n} ; z ≡ exp ( - β ℏ ω ) ; β ≡ \frac{1}{kT}$$

The average internal energy can be computed. The zero-point energy is suppressed in the rest of the statistical computation of the partition function

$$Q ≡ {\sum }_{n = 0}^{∞} {z}^{n} = \frac{1}{1 - z} = \frac{1}{1 - exp ( - β ℏ ω )}$$

for all possible eigenstates.

$${〈 E 〉}_{Q} = \frac{\sqrt{z} {\sum }_{n = 0}^{∞} {E}_{n} W ( {E}_{n} )}{\sqrt{z} {\sum }_{n = 0}^{∞} W ( {E}_{n} )} = \frac{- \frac{\partial Q}{\partial β}}{Q} = - \frac{\partial Log Q}{\partial β} = \frac{1}{2} ℏω + \frac{ℏω}{exp ( \frac{ℏω}{{k}_{B} T} ) - 1} \qquad ( 7 )$$

where a half quanta, ½ [symbol] ω, is understood to keep an even symmetric eigenstate wave-function for the symmetric BB walls. Given the density of states (dos) in wavelength, Planck spectral density U_{Planck} was re-derived:

$${U}_{Planck} ( λ \vert T ) = \frac{8 π {C}_{o} h}{{λ}^{5}} \cdot [ \frac{1}{exp ( \frac{{hC}_{o}}{{k}_{B} T λ} ) - 1} ] \qquad ( 8 )$$

where mode volume is

$${V}_{λ} ≡ 2 x \frac{4 π}{3} {\vert \frac{1}{λ} \vert}^{3} and dos ≡ \frac{d {V}_{k}}{d λ} ≡ \vert - \frac{8 π}{{λ}^{4}} \vert ;$$

and the photon energy

$$ℏ ω = hf = \frac{{hC}_{o}}{λ}$$

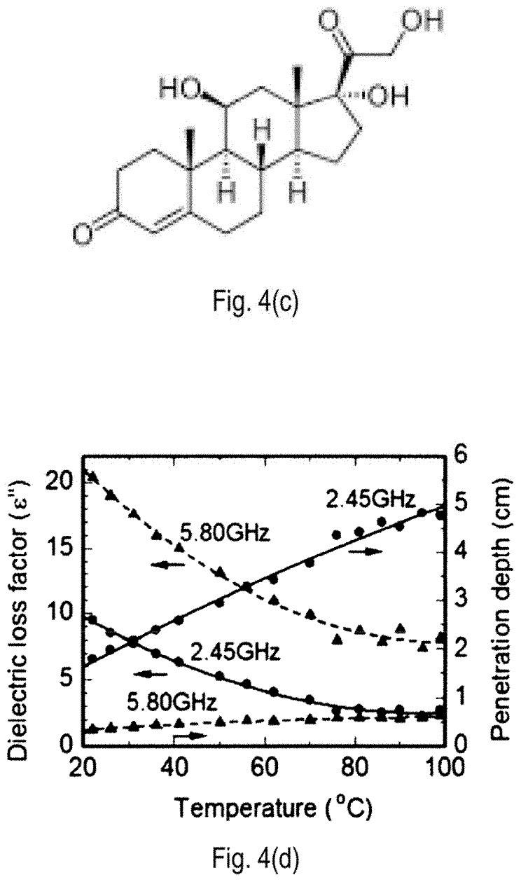

Planck's constant h is found by fit experiments to be 6.62606957×10⁻³⁴ Joule-second and Boltzmann's constant k_{B}=1.3806504×10⁻²³ J/K. FIG. 5 shows the Planck black-body radiation spectral density, Eq (8), results in a typical long wavelength decay in the inverse 5^{th} power law in the wavelength λ.

### Kelvin Temperature Determination:

To determine the temperature value of the radiation spectrum, the peak of Planck spectral law with respect to the wavelength is determined. Let a≡hCₒ/k_{B}. Then, the slope is computed and set to zero in order to numerically obtain Wien's displacement law

$$\begin{matrix}8 π {C}_{o} h \frac{d}{d λ} ( \frac{1}{{λ}^{5}} \cdot [ \frac{1}{exp ( \frac{a}{T λ} ) - 1} ] ) = 0 & ( 9 a ) \\ {λ}_{max} T = \frac{a}{5} ( \frac{1}{1 - exp ( - \frac{a}{T λ} )} ) ; \\ {λ}_{max} T \approx 2.8 {10}^{- 3} meter Kelvin & ( 9 b )\end{matrix}$$

In fact, the formula is true near the flat spectrum of longer LWIR in FIG. 5.

Near the homeostasis temperature 27° C.=300° K the emissive radiation spectrum peaks LWIR 10 μm wavelength at the 28 GHz microwave region. Thus, if the microwave backscattering happens to cover the peak spectrum, one can easily determine the radiation source Kelvin temperature from numerically matching with the slope (9a) and the curvature of (9b) of the Planck spectral values. For example, for human homeostasis at 37° C.=310° K, the peak is shifted slightly to a shorter LWIR, 10×31/30≈9.4 μm. Once the source temperature is determined, one can analytically continue the same factor to all other components along the Planck spectral curve temperature parameter, resulting in a consistent human visual system (HVS) color display.

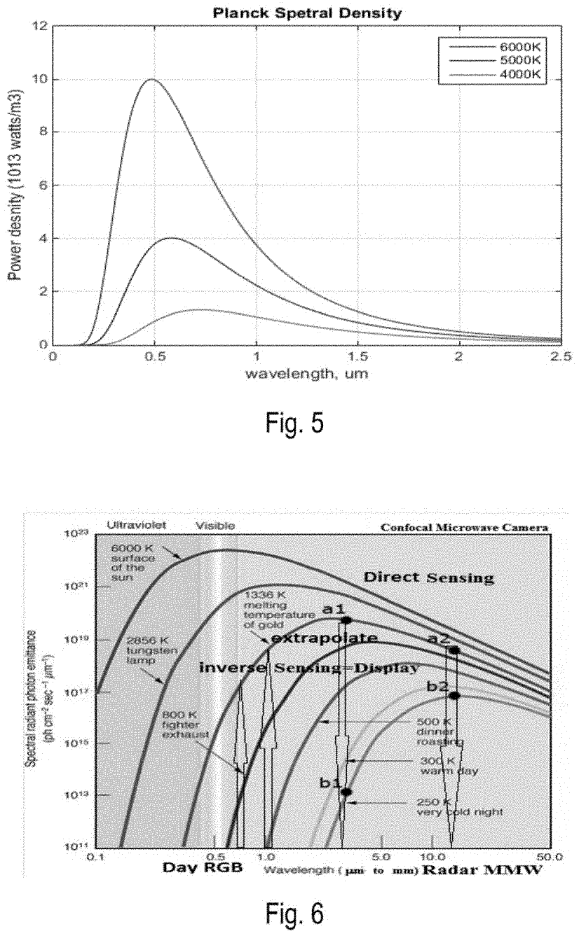

As shown in FIG. 6, an analytic continuation after BSS and selective projection of desired targets by means of millimeter wave radar CTMI in terms of Boltzmann entropy theory along the Planck spectral curve for any portion of measuring spectral shape to determine the unique Kelvin temperature per spectral curve.

### From Traditional Imaging to Modern Compressive Imaging

Due to the availability of massive parallel & distributed semiconductor arrays, traditional sensing and displaying tend to be overkill in the sense of an information content point of view. It would be beneficial in a separate optimum approach to the sensing and displaying to achieve also critical sampling in the Nyquist sense. Preferably this is done at the sensor domain, not at the display domain, where wasteful redundant measurements have already been performed. Then, post-processing uses the compression standard (JPEG (DCT) JPEG 2000 Wavelet Transform). Recently, Candes (of Caltech), Romberg (of GIT), Donohoe (of Stanford) and Tao (of UCLA), have jointly developed a technique for compressive sensing (CS) known as CRDT CS, operated at the sensor domain in terms of L1 norm (for x≠0; x⁰=1 meaning sampling; otherwise x=0, with 0⁰→0 defined for zero, not sampling). The sparseness degree s should be bounded between the pixel size N and m linear combination readouts; namely N>>s>>m>>0.

The application in CTMI for medical screening of cancers has the zero-range contact imaging at patient skin that requires a lower intensity of microwave, but longer time integration from seconds to minutes because the fine pinhole dense array is in contact with patient skin, while the patient breathes in a gentle motion. Thus, automatic motion compensation [8] [9] [10] has to be built in at the sensor domain of CTMI. However, traditional motion compensation depends on neighborhood change detection which may or may not work when the neighborhood imaging point disappears [11,12]. Nevertheless, compressive sensing less at sensor domain will be a good strategy for a patient's psychology. It would be beneficial to replace x-ray mammograms with CTMI for routine household cancer screening. This works because malignant tumors exhibit dense tissue with high dielectric constants compared to normal breast tissue. Only frequent change detection can provide growth rate analysis comparable to a culture biopsy. Thus, repeated screening with a much reduced accumulated radiation exposure is necessary to screen at early stages of tumor growth (that is, aforementioned 4 stages of cancer: neo-angiogenesis, extraversion, transportation, and metastasis). This is possible with the advent of nano-pulse microwaves, which can be used for dielectric vibration reemitted EM imaging. CS L1 norm is presented in the context of familiar L2 norm classical image processing as follows.

### Classical LMS Wiener Filter

Beginning with a classical LMS Wiener filter imaging model, assuming a linear space-invariant multi-spectral frequency-f white-noisy imaging f,k equation:

I_{f,k}=S_{f,k}O_{f,k}+N_{f,k} (10a)

where double indices indicate spectral f and the Fourier de-convolution transform (\vec{x}→\vec{k}) in the product form between the point-spread function S_{f,k}, unknown object O_{f,k}, and white noise:

<N_{f,k}N_{f′,k′}*⁺>=|N|²δ_{f,f′}δ_{k,k′}. (10b)

Supervised LMS Wiener filter estimated object is:

Ô=WI

In order to divide by a non-zero denominator, both sides are multiplied by the Hermitian conjugate image:

$$〈 \hat{O} {I}^{* +} 〉 = W 〈 {II}^{* +} 〉 \\ {W}_{f , k} = \frac{〈 \hat{O} {I}^{* +} 〉}{〈 {II}^{* +} 〉} = \frac{{S}^{* +} 〈 {OO}^{* +} 〉}{{\vert S \vert}^{2} 〈 {OO}^{* +} 〉 + {\vert N \vert}^{2}} = {S}^{- 1} {\{ 1 + {ɛ}^{- 1} \}}^{- 1} = \{ \begin{matrix}{S}^{* +} \\ {S}^{- 1}\end{matrix} \} ; \\ SNR ɛ = \frac{{\vert S \vert}^{2} 〈 {OO}^{* +} 〉}{〈 {\vert N \vert}^{2} 〉} ≶ 1 \qquad ( 11 )$$

where, in the strong SNR ε>1, the Wiener filter is reduced to the inverse filtering S⁻¹ as the noise can be ignored. On the other hand, for a weak SNR, there is the self-matched filter S*⁺.

### Vander Lugt Match Filter

For the Vader Lugt match filter, a general proof shows that two vectors (\vec{X}, \vec{Y}) will be matched when their inner product (\vec{X}∘\vec{Y}) is maximal, resulting in error minimization between \vec{X} and \vec{Y}.

|\vec{X}−\vec{Y}|²=|\vec{X}|²+|\vec{Y}|²−2\vec{X}∘\vec{Y}

Thus, self-matched filters require \vec{X}∥\vec{Y} in order to maximize \vec{X}∘\vec{Y}.

### Real-time Aided Target Recognition (AiTR)

The inventive approach to compressive sampling at surveillance is possible because it is first established to create by BSS utilizing LCNN similar to the super-mother wavelet theorem [7] for non-real time library of template construction at the same local surveillance site, before forming a linear combination for subsequently real-time surveillance library of sources, and the library of templates Ô_{f,k}. A real-time linear combination S_{f,k} can then be formed with these templates to estimate in an LMS sense, but overcome the usually non-real time library approach with L2 norm and compressive sensing with L1 norm as follows:

min. ∥I_{f,k}−Σ_{f,k}S_{f,k}Ô_{f,k}∥²+λ₁∥Ô_{f,k}∥² (12a)

While infinitely large 2-D library templates can in principle match any 2-D image, it is computationally intractable and therefore is not admissible. Therefore, it is necessary to impose the number of library templates functions to be used for the LMS optimization in terms of Lagrange parameter λ₁.

### CRDT Compressive Sensing

CRDT CS [13,14,15] operates at the pre-processing sensor domain in L1 norm (x°=1). This L1 operation is operated at the sensor domain, as compressive sensing [4]:

min. ∥I_{f,k}−Σ_{f,k}S_{f,k}Ô_{f,k}∥²+λ₁∥Ô_{f,k}∥²+λ₂|S_{f,k}|¹ (12b)

The estimated malignant-to-normal breast tissue ratio of dielectric constants (N. Epstein/U. Calgary) defined by Maxwell vector displacement current D=ϵE) is between 2:1 and 10:1, depending on the density of the normal tissue.

### REFERENCES

[1] Harold Szu and Charlie Hsu, “Landsat Spectral De-mixing a la Super-resolution of Blind Matrix Inversion by Constraint Max-Ent Neural Nets”, Proc. of SPIE, vol. 3078, pp. 147-160, 1997.
[2] Harold H. Szu, James R. Buss, Ivica Kopriva, “Early breast tumor and late SARS detections using space-variant multispectral infrared imaging at a single pixel,” Proc. SPIE. 5439, Independent Component Analyses, Wavelets, Unsupervised Smart Sensors, and Neural Networks II 116 (Apr. 12, 2004)
[3] Harold Szu, Lidan Miao, and Hairong Qi, “Unsupervised Learning with Mini Free Energy”, Proc. of SPIE, vol. 6576, pp. 657605-1, 2007.
[4] C Y Lee, H Y Hsieh, S C Lee, C S Huang, Y C Chang, C M Chen, H H Szu, “Spatiotemporal sharpening of sub-pixel super-resolution by means of two infrared spectrum cameras for early cancer detection,” Proc. SPIE. 6979, Independent Component Analyses, Wavelets, Unsupervised Nano-Biomimetic Sensors, and Neural Networks VI 69790R (Apr. 3, 2008)
[5] Harold Szu, Philip Hoekstra, Joseph Landa, Nadarajen A. Vydelingum “Neo-Angiogenesis Metabolic Biomarker of Tumor-genesis Tracking By Infrared Joystick Contact Imaging in Personalized Homecare System,” Proc. of SPIE Vol. 9118, 91180T (2014)
[6] Harold Szu, “Thermodynamic Learning Rule”, Natural Intelligence: the INNS Magazine, vol. 2(1), pp. 19-27, 2013.
[7] Jae H. Cha, A. Lynn Abbott, Harold H. Szu, Jefferson Willey, Joseph Landa, Keith A. Krapels “Neuromorphic Implementation of a Software-defined Camera that can “See” through Fire and Smoke in Real-time,” SPIE Proc. 9118-7 2014.
[8] Jae H. Cha, A. Lynn Abbott, Keith A. Krapels, and Harold H. Szu, “Theory of Enhancing Thermal Imaging through Fire”, Proc. SPIE, vol. 9118, 2014.
[9] Harold H Szu & Brian A. Telfer, “Mathematics of adaptive wavelet transforms: relating continuous with discrete transforms Opt. Eng. V. 33(7):2111-2124 (1994)
[10] Harold H. Szu and J. A. Blodgett,” Self-reference spatiotemporal image-restoration technique JOSA, Vol. 72, Issue 12, pp. 1666-1669 (1982);
[11] Harold Szu, J. P. Garcia, “Radar jitter suppression by self-reference matched filter,” Electronics Letters V. 29, pp. 1045-1046 07/1993; DOI: 10.1049/
[12] Harold Szu, Charles Hsu, Jeffrey Jenkins, Jefferson Willey, Joseph Landa, “Capturing significant events with neural networks,” Neural Networks V. 29-30 (2012) pp. 1-7
[13] Harold Szu, Charles Hsu, Jeffery Jenkins, Binh Trans, Joe Landa, “Smartphone EEG,” Computational Intelligence (2014)?
[14] X. Li, S. K. Davis, S. C. Hagness, D. W. van der Weide, and Barry D. Van Veen, “Microwave Imaging via Space-Time Beamforming: Experimental Investigation of Tumor Detection in Multilayer Breast Phantoms,” IEEE Trans. MWT, V. 52 (8), pp. 1856, 1865, 2004
[15] Q. Fang, P M Meaney, K D Paulsen, “Microwave image reconstruction of tissue property dispersion characteristics utilizing multiple-frequency information,” IEEE Trans. MWT, V. 52(8), pp. 1866-1875 (2004).
[16] P M Meaney, M W Fanning, R M di Florio-Alexander, P A Kaufman, S D Geimer, T Zhou, K D Paulsen, “Microwave Tomography in the Context of Complex Breast Cancer Imaging,” IEEE Eng Med Biol Soc. Conf. Proc. 2010, pp. 3398-3401 (2010)
[17] R. N. Lawson, “Implications of Surface Temperatures in the Diagnosis of Breast Cancer,” Can Med Assoc J V75: pp. 309-310 (1965).
[18] R. N. Lawson, & M. S. Chughtai, “Breast cancer and body temperatures,” Can Med Assoc. J V88: pp. 68-70 (1963).
[19] Manu Mital, “Thermal Detection of Embedded Tumors using Infrared Imaging,” VPI&SU Thesis (with Elaine P. Scott, Chair), Mech. Eng. Aug. 23 (2004).
[20] J. Gershen-Cohen, J. Haberman, and E. E. Brueschke, “Medical thermography: A summary of current status,” Radiol Clin North Am V. 3: pp. 403-431 (1965).
[21] IEEE Eng. Med. & Bio. “Infrared: from Tanks to Tumors,” V21 (No. 6), N. A. Diakides (ed.); J. Pail & J. Lupo; J. M. Irvine; B. F. Jones & P. Plassmann; K. Otsuka, S. Okada, M. Hassan, T. Togawa; I. Pavlidis, J Levine; N. Katuta, S. Yokoyama, K. Mabuchi; A. Merla, L. D. Donato, S. D. Luzio, G. Farina, S. Pisarri, M. Peoietti, F. Salsano, G. L. Romani; J. F. Head, R. J L. Elliott; A. Merla, L. D. Donato, S. D. Luzio, G. L. Romani; pp. 32-91 (November-Dec. 2002)
[22] K. Mabuchi, & S. Mizushina, B. Harrison, “Adv. Tech. & Clin. App. in Biomed. Thermology,” Harwood Acad. Publ. (Tokyo, 1994) pp. 1-319.
[23] Harold Szu, Charles Hsu, Gyu Moon, Takeshi Yamakawa, Binh Q. Tran, Tzyy Ping Jung, and Joseph Landa, “Smartphone Household Wireless Electroencephalogram Hat” Applied Computational Intelligence and Soft Computing, Vol. 2013, Article ID 241489, pp. 8 pages (2013).
[24] S A Curley, F Palalon, K E Sanders, N V Koshkina, “The effect of noninvasive rf treatment & hyperthermia on malignant and nonmalignant cells,’ Int. J. Envion. Res. Public Health, V11, pp. 9142-9153 (2014)
[25] S Horikoshi, N. Serpone, “microwave frequency effects in organic chemistry,” mini-review in organic chemistry, V8, pp. 299-305 (2011).
[26] C Gabriel, S Gabriel, E H Grant, B S J Halstead, D M P Mingos, “Dielectric parameters relevant to microwave dielectric heating,” Chemical Society Reviews, v 27, pp. 213, 223 (1998)
[27] A. R. von Hippel, Dielectric Materials and Applications, MIT Press, Cambridge, Mass., USA, 1954; A. R. von Hippel, Dielectric and Waves, MIT Press, MIT, Cambridge, Mass. USA, 1954.

I claim:

1. A passive thermal spectral imaging camera, comprising an active confocal tunable microwave illuminator tuned to human physiology bio-molecules.

2. The camera of claim 1, further comprising an active microwave radar and a passive electro-optic-infrared camera.

3. The camera of claim 1, further comprising a thermal biomarker, wherein the thermal biomarker includes a microwave oven configured to operate at about 2.4 GHz., a microwave-mixer configured for upshift and/or downshift, and a beam expander.

4. The camera of claim 3, wherein the beam expander includes a metallic pin-hole array.

5. The camera of claim 4, wherein the pin-hole array includes apertures having a diameter of about 20 cm.

6. An imaging system, comprising:
a passive electro-optic-infrared camera; and
a thermal biomarker.

7. The system of claim 6, further comprising:
a microwave oven; and
a microwave mixer;
wherein the thermal biomarker includes a confocal tunable microwave illuminator, which includes a waveguide coupled to a magnetron of the microwave oven and to the microwave mixer.

8. The system of claim 7, wherein the microwave mixer is configured to downshift and/or upshift an output of the magnetron.
